(19) United States
(12) Patent Application Publication
(10) Pub. No.: US 2022/0178914 A1
(43) Pub. Date: Jun. 9, 2022

(54) LITHIUM HEPARIN AS A BLOCKING AGENT

(71) Applicant: Ortho-Clinical Diagnostics, Inc., Raritan, NJ (US)

(72) Inventors: Brett Hirsch, Raritan, NJ (US); Paul Contestable, Raritan, NJ (US)

(21) Appl. No.: 17/539,903

(22) Filed: Dec. 1, 2021

Related U.S. Application Data
(60) Provisional application No. 63/121,161, filed on Dec. 3, 2020.

(51) Int. Cl. G01N 33/53; G01N 33/569

(52) U.S. Cl. CPC G01N 33/5306; G01N 2469/10; G01N 2333/02; G01N 33/56983

(57) ABSTRACT

Described herein are blocking agents that can be used in diagnostic assays to prevent false positive results.

### CROSS REFERENCE TO RELATED APPLICATIONS

This application claims the benefit of U.S. provisional patent application No. 63/121,161, filed Dec. 3, 2020, the entire disclosure of each is incorporated herein by reference.

### FIELD

Described herein are blocking agents used in immunodiagnostic assays, related devices and systems, and methods of using the same.

### SUMMARY

Described herein are blocking agents that can be used in diagnostic assays. In some embodiments, the diagnostic assays can be immunoassays.

The blocking agents described can reduce a rate of false positive results detected in a patient population.

The blocking agent can be added to a reagent mixture or added as a reagent during an assay.

In one embodiment, a blocking agent can include heparin. In other embodiments, a blocking agent can include lithium. In some embodiments, the blocking agent is lithium heparin.

In some embodiments, provided are immunoassay reagents which include one or more analyte binding agents in a diluent, and a blocking agent in an amount sufficient to reduce non-specific binding in an assay of a sample for the analyte.

Further provided is a sample composition which comprises a sample to be assayed for the presence of an analyte, an analyte binding agent, and a blocking agent in an amount sufficient to reduce non-specific binding in an assay of the sample for the analyte.

Also provided are methods of detecting an analyte in a sample, using a blocking agent to reduce non-specific binding. The methods comprise combining a sample to be analyzed for the presence of an analyte with a blocking agent and one or more analyte binding agents, so as to form a complex of any analyte present in the sample and the analyte binding agent(s), wherein the blocking agent reduces non-specific binding, and detecting the resulting complex so as to detect the analyte.

### BRIEF DESCRIPTION OF THE DRAWINGS

FIG. 1 is a general flow chart of an assay as described herein.

FIG. 2 is a graph showing assays run with and without lithium heparin.

FIG. 3 is a graph showing retention of classification of samples that include lithium heparin.

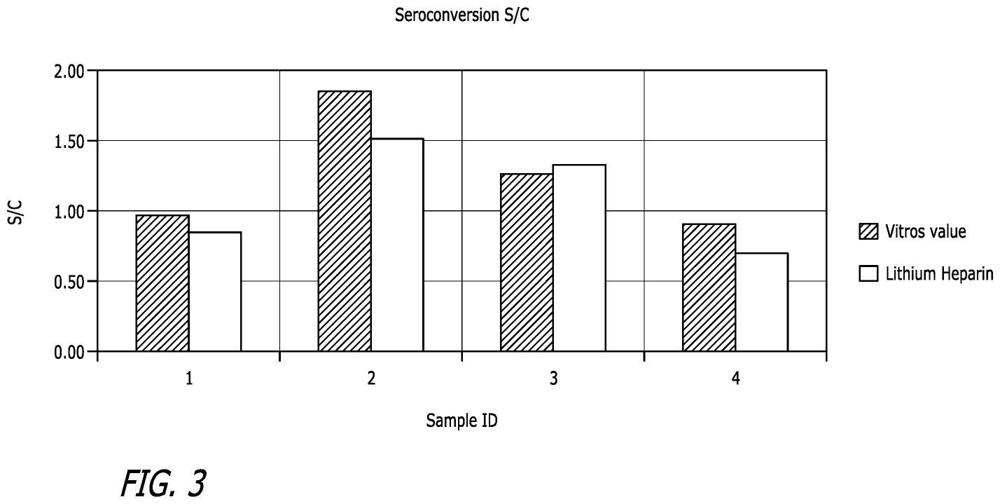

### DETAILED DESCRIPTION

Described herein are blocking agents that can be used in diagnostic assays to prevent false positive results. In some embodiments, the diagnostic assays can be immunoassays. In some embodiments, provided are compositions and systems including an immunoassay reagent which comprises one or more analyte binding agent(s) in a diluent, and a blocking agent in an amount sufficient to reduce non-specific binding in an assay of a sample for the analyte.

In some embodiments, the diagnostic assays and/or immunoassays described herein can be performed in any manner that achieves a result. In some embodiments, the diagnostic assays and/or immunoassays can be performed as a benchtop assay. In other embodiments, the diagnostic assays and/or immunoassays can be performed in an automated or semi-automated system. In one embodiment, the diagnostic assays and/or immunoassays can be run on a mainframe analyzer. In some embodiments, the diagnostic assays and/or immunoassays can be in vitro tests.

It is often desirable to determine the presence and the concentration of analytes in biological specimens or samples. An analyte is a substance or chemical constituent that is determined in an analytical procedure (such as an immunoassay). Immunoassays are based on the concept of binding partners. An analyte of interest binds to an analyte binding agent (such as, for example, an antibody to the analyte, or a receptor for the analyte), and the analyte and the analyte binding agent are thus referred to as “binding partners.”

The sample to be analyzed for the presence of analyte can be any suitable sample, preferably a blood sample such as a serum sample or plasma sample. Blood plasma is the liquid component of blood in which the blood cells are suspended. A simple way to separate plasma from blood cells in a blood sample is by centrifugation. Serum refers to blood plasma in which clotting factors have been removed naturally by allowing the blood to clot prior to isolating the liquid component. Plasma samples can be obtained from blood tubes which contain anticoagulants such as sodium heparin, sodium citrate, sodium fluoride, and potassium oxalate or potassium EDTA (ethylenediamine tetraacetic acid). In the case of a plasma sample, the plasma can be obtained using an anticoagulant.

In many immunoassays, the analyte binding agent is an antibody. Such antibodies are often provided in a diluent such as buffer. The antibody may be of any immunoglobulin class, including, for example, IgG or IgM. The antibody may be a monoclonal antibody or a polyclonal antibody. Analytes of interest present in a sample can bind to immobilized capture antibodies, and then labeled antibody or antibodies in turn bind to the captured analyte. The label may be any known in the art, and include, for example, horseradish peroxidase and alkaline phosphatase. A detected signal is then indicative of an amount of analyte present in a sample. The method of detection can depend upon the type of label chosen and can include calorimetric, fluorometric, or chemiluminescent methods.

Also, described are methods of reducing false positive rates, or improve specificity, for immunoassays. In one embodiment, the reduction can be for the detection of Hepatitis C antibodies. The addition of heparin (e.g. lithium heparin) to the assay's reagents can accomplish this. Prevention of false positive results can be a result of preventing a non-specific binding interaction.

In some embodiments, the methods, compositions, and systems can be used for the in vitro qualitative detection of immunoglobulin G antibody to hepatitis C virus (anti-HCV) in human serum and/or plasma. In some embodiments, the detection is using VITROS ECi/ECiQ/3600 Immunodiagnostic Systems or VITROS 5600/XT 7600 Integrated Systems.

In some embodiments, the methods, compositions and systems can be used in lateral flow assays, such as on slides or dry slides. In such embodiments, the blocking agent(s) can be added to the slide before or during analysis. In some embodiments, the blocking agent can be added as a coating in a channel or well within or on a slide.

In some embodiments, the methods, compositions, and systems can be used in point of care systems wherein the analysis of human serum and/or plasma is desired.

In some embodiments, the methods, compositions, and systems can be used in automated analyzers.

In some embodiments, the blocking agent can be added to a lateral flow assay slide or chip as needed in order to run a sample.

Assay results, in conjunction with other laboratory results and clinical information, can be used to provide evidence of infection with hepatitis C virus in persons with signs or symptoms of hepatitis and in persons at risk for hepatitis C infection. Further, the assays as described herein may be used to screen for hepatitis C infection in pregnant women to identify neonates who are at high risk of acquiring HCV during the prenatal period.

The hepatitis C virus (HCV) is now known to be a causative agent for most, if not all, blood-borne non-A, non-B hepatitis (NANBH). Studies indicate that HCV is transmitted through contaminated blood and blood products, through blood transfusions or through other close, personal contacts. The presence of anti-HCV indicates that an individual may have been infected with HCV and may be capable of transmitting HCV infection. Three recombinant hepatitis C virus encoded antigens (c22-3, c200 and NS5) can be used to detect HCV. The recombinant protein c22-3 is encoded by the putative core region of the HCV genome. HCV recombinant protein c200 is encoded by the putative NS3 and NS4 regions of the HCV genome. The c200 protein contains the c33c protein sequence which is genetically linked to the c100-3 protein sequence. Studies have indicated that antibodies which develop after infection with HCV are often reactive with c22-3 and/or c33c. HCV recombinant protein NS5 is encoded by the putative NS5 region of the HCV genome. A significant proportion of persons infected with HCV develop antibodies to NS5. The host organism for all three HCV recombinant antigens is S. cerevisiae (yeast).

In some embodiments, the compositions used for detection can include three recombinant hepatitis C virus encoded antigens. In other embodiments, one or two can be used. In some embodiments, c22-3, c200 and NS5 are used. In other embodiments, c22-3 and c200, or c22-3 and NS5, or c200 and NS5 are used. In still other embodiments, c22-3, c200 or NS5 is used.

Hepatitis C (HCV) often affects the liver and is a major concern for both patient care and the world's blood supply. This infection produces HCV antibodies in a patient's blood that can be captured and detected by diagnostic immunoassays. These immunoassays play a key role in identifying infected patients and blood samples. While these assays are designed to be very sensitive and specific, false positive results are a reality. A false positive result occurs when a diagnostic tool such as an immunoassay gives a positive result when there is, in fact, no HCV antibodies present in a test sample. These false positive results can occur for a variety of reasons including, but not limited to, non-specific binding.

Non-specific binding occurs when a compound in a sample is captured and detected by an assay when that compound is not the target analyte (i.e. not the HCV antibody). A blocking agent as described herein can be included in assay reagent formulations to minimize these false positives.

The blocking agents described can reduce a rate of false positive results detected in samples and/or over an entire patient population.

The blocking agent can be added to a reagent mixture or added as a reagent during an assay.

In one embodiment, a blocking agent can include a glycosaminoglycan, such as but not limited to heparin, chondroitin, dermatan, keratin, and hyaluronic acid. In one embodiment, the glycosaminoglycan is heparin. That heparin can be coordinated with a metal, such as a metal ion. That metal can be an alkali metal such as a Group I element. Metals can include lithium, sodium, potassium, rubidium, francium, or cesium.

In some embodiments, the blocking agent can include lithium. In some embodiments, the blocking agent is lithium heparin.

Lithium heparin is a negatively charged polysaccharide polymer classified as a glycosoaminoglycan and is produced and stored within mammalian tissues.

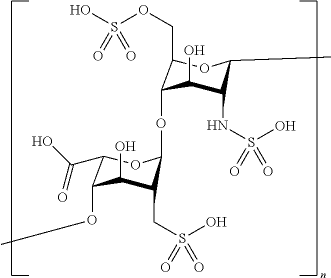

The heparin as described herein can have a chain length that results in reduced false positives. In some embodiments, n can be 1-100, 1-1,000, or 1-10,000.

Heparin has been utilized in medicine as an anticoagulant as it interacts with several cofactors within the human coagulation process. Lithium heparin is the lithium salt of heparin.

The blocking agent is provided in an amount sufficient to reduce non-specific binding in an assay of a sample for the analyte. When the blocking agent is lithium heparin, the amount is preferably from about 0.25 mg/mL to about 4 mg/mL (equivalent to about 0.025% to about 0.4%).

In some embodiments, an immunometric technique is used. Such a technique involves a two-stage reaction. In the first stage, HCV antibody present in the sample binds with HCV recombinant antigens coated on the wells. Unbound sample is removed by washing. In the second stage, horseradish peroxidase (HRP)-labeled antibody conjugate (mouse monoclonal anti-human IgG) binds to any human IgG captured on the well in the first stage. Unbound conjugate is removed by washing.

The bound HRP conjugate is measured by a luminescent reaction. A reagent containing luminogenic substrates (a luminol derivative and a peracid salt) and an electron transfer agent, is added to the wells. The HRP in the bound conjugate catalyzes the oxidation of the luminol derivative, producing light. The electron transfer agent (a substituted acetanilide) increases the level of light produced and prolongs its emission. The light signals are read by the system. The amount of HRP conjugate bound is indicative of the level of anti-HCV present in the sample.

A reagent composition can include a blocking agent as described herein in combination with diagnostic regents in a buffer. In some embodiments, those diagnostic reagents are immunodiagnostic reagents.

In other embodiments, a reagent composition can include a blocking agent as described herein, a labeled protein, and antigens in buffer.

In other embodiments, a reagent composition can include a blocking agent as described herein, a HRP-labeled protein, and antigens in buffer.

In other embodiments, a reagent composition can include a blocking agent as described herein, a HRP-labeled protein, an antimicrobial agent, and antigens in buffer.

In other embodiments, a reagent composition can include a blocking agent as described herein, a HRP-labeled protein, and HCV encoded antigens in buffer.

In other embodiments, a reagent composition can include a blocking agent as described herein, a HRP-labeled protein, an antimicrobial agent, and HCV encoded antigens in buffer.

In other embodiments, a reagent composition can include a blocking agent as described herein, a HRP-labeled IgG, and HCV encoded antigens in buffer.

In other embodiments, a reagent composition can include a blocking agent as described herein, a HRP-labeled IgG, an antimicrobial agent, and HCV encoded antigens in buffer.

In other embodiments, a reagent composition can include lithium heparin, a labeled protein, and antigens in buffer.

In other embodiments, a reagent composition can include lithium heparin, a HRP-labeled protein, and antigens in buffer.

In other embodiments, a reagent composition can include lithium heparin, a HRP-labeled protein, an antimicrobial agent, and antigens in buffer.

In other embodiments, a reagent composition can include lithium heparin, a HRP-labeled protein, and HCV encoded antigens in buffer.

In other embodiments, a reagent composition can include lithium heparin, a HRP-labeled protein, an antimicrobial agent, and HCV encoded antigens in buffer.

In other embodiments, a reagent composition can include lithium heparin, a HRP-labeled IgG, and HCV encoded antigens in buffer.

In other embodiments, a reagent composition can include lithium heparin, a HRP-labeled IgG, an antimicrobial agent, and HCV encoded antigens in buffer.

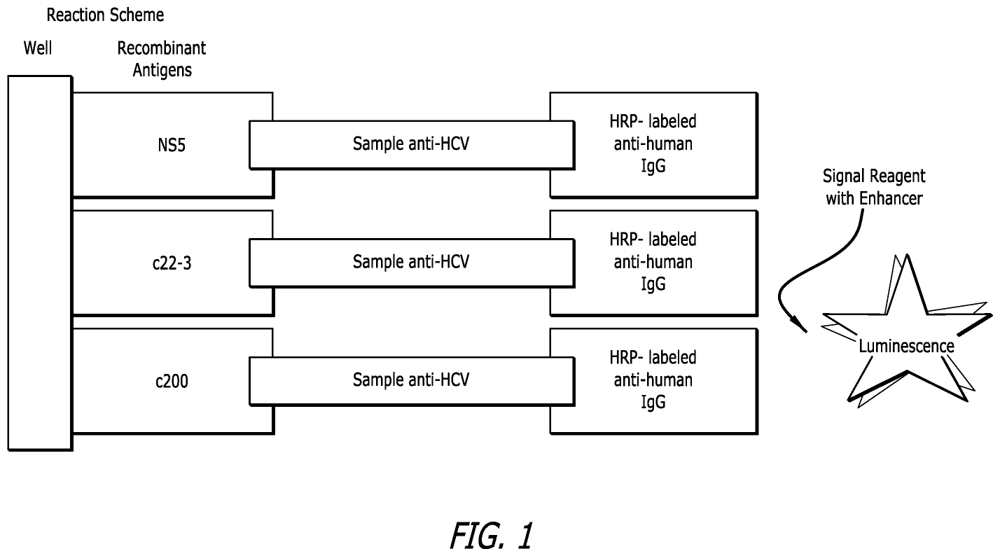

As described, three recombinant hepatitis C virus encoded antigens (c22-3, c200 and NS5) can be used to detect HCV. When used, a reaction scheme similar to that in FIG. 1 is used, wherein each of the three antigens are measured using HRP labeled anti-human IgG.

In some embodiments, reagents used to detect HCV can include Hepatitis C virus recombinant antigens (NS5, c22-3, c200) derived from yeast (S. cerevisiae), reagent (buffer with 2-chloroacetamide anti-microbial agent), conjugate reagent (HRP-mouse monoclonal anti-human IgG, 1.04 ng/well) in buffer with anti-microbial agent (1% ProClin 300 w/w), and lithium heparin.

In some embodiments, reagents used to detect HCV can include Hepatitis C virus recombinant antigens (NS5, c22-3, c200) derived from yeast (S. cerevisiae), 18.2 mL assay reagent (buffer with 2-chloroacetamide anti-microbial agent), 20.6 mL conjugate reagent (HRP-mouse monoclonal anti-human IgG, 1.04 ng/well) in buffer with anti-microbial agent (1% ProClin 300 w/w), and lithium heparin.

In some embodiments, the Hepatitis C virus recombinant antigens (NS5, c22-3, c200) can be coated on wells, such as wells in a 100 well plate.

In some embodiments, the reagent packs including the blocking agent are kept refrigerated in order to preserve stability.

In some embodiments, inclusion of heparin, such as lithium heparin, in the assay reagent for an HCV immunoassay can markedly reduce the occurrence of false positives. Heparin may bind to a compound within a serum or plasma sample that, when unbound, can be the cause of a non-specific binding events, leading to false positive assay results. The addition of heparin, including lithium heparin, into the formulation of an immunodiagnostic assay's reagents is able to reduce the incidence rate of false positives, increasing the assay's overall specificity.

In some embodiments, including lithium heparin in an assay can reduce false positive rates by greater than about 70%, greater than about 80%, greater than about 85%, greater than about 86%, greater than about 90%, or greater than about 95%.

In some embodiments, lithium heparin does not impact the sensitivity of assays.

In some embodiments, lithium heparin does not affect the assay's ability to detect true positive samples.

In some embodiments, lithium heparin can increase the specificity of an assay. In some embodiments, specificity can increase by greater than about 0.05%, greater than about 0.1%, greater than about 0.2%, greater than about 0.3%, greater than about 0.4%, or greater than about 0.5%.

Also provided are methods of detecting an analyte in a sample comprising: combining a sample to be analyzed for the presence of an analyte with a blocking agent and an analyte binding agent, so as to form a complex of any analyte present in the sample and the analyte binding agent, wherein the blocking agent reduces non-specific binding in the method; and detecting the resulting complex so as to detect the analyte. In one embodiment, the sample is combined with the blocking agent and the resulting sample is then combined with the analyte binding agent. In another embodiment, the sample is combined with the analyte binding agent and the resulting sample is then combined with the blocking agent, in yet another embodiment, the analyte binding agent is provided as an immunoassay reagent comprising the analyte binding agent in a diluent and the blocking agent, and the immunoassay reagent is combined with the sample.

### Example 1

Lithium heparin is added to an assay reagent of a VITROS Immunodiagnostic Product HCV assay at a concentration of 0.04 to 0.10% w/v. The assay is then used to analyze a series of 112 samples known to give a false positive result when using this assay. These samples previously tested as positive on the VITROS HCV assay, but are then proven to be negative by immuno-blot and/or nucleic acid testing (NAT). Of these 112 samples, only 16 continue to give a false positive result once lithium heparin is added to the assay's reagents.

FIG. 1 illustrates results of this study. The x-axis represents sample ID number and the y-axis represents the signal-to-cutoff value (S/C). Any sample with a result >1.0 S/C is considered positive in this assay, the VITROS HCV assay. The control data set are the results given on the unmodified assay, while the lithium heparin data points are the result of the same sample when the assay contains the lithium heparin additive in the assay reagent. This suggests that addition of lithium heparin reduces the false positive rate.

### Example 2

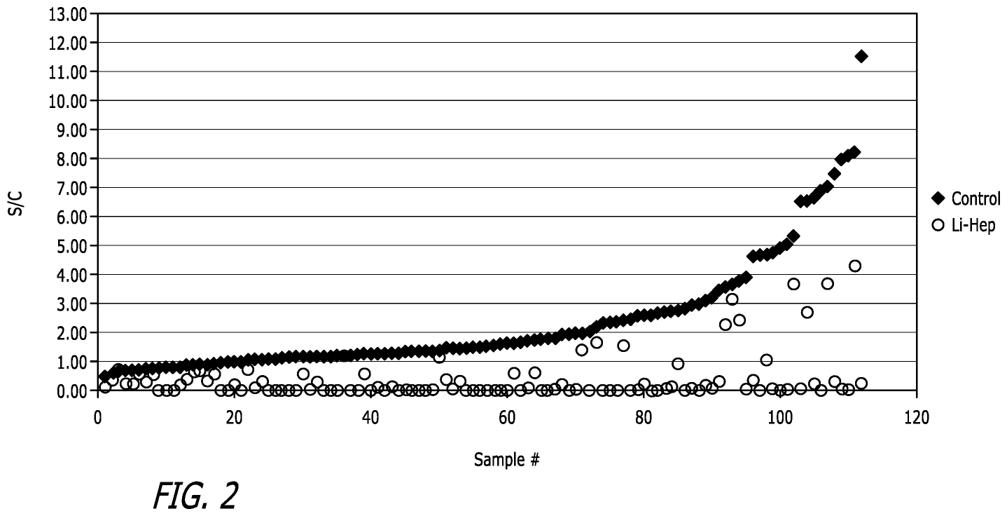

Lithium heparin does not significantly impact the sensitivity of assays. In other words, it does not affect the assay's ability to detect true positive samples. Four seroconversion panel members (samples collected from a recently infected donor during the development an initial immune response) near the 1.0 S/C cutoff for a positive result are investigated. The results are illustrated in FIG. 2.

The left bar result is that given by the on-market VITROS HCV assay, and the right bar result is this assay with the lithium heparin additive. The addition of heparin did not change the classification of any of these four positive samples.

### Example 3

The addition of lithium heparin can increase the specificity of an assay. One thousand presumed negative patient samples are run on an on-market VITROS HCV assay and then again on the same assay with the lithium heparin additive. Specificity increases from 99.80% to 99.90%. This assay and similar diagnostic products are used to screen millions of patients and blood samples each year. This reduction in false positives has the potential to correct tens of thousands of results annually.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

I claim:

1. An immunoassay reagent comprising: one or more binding agent(s) in a diluent; and lithium heparin in an amount sufficient to reduce non-specific binding in an assay.

2. The immunoassay reagent of claim 1, wherein the lithium heparin is present in an amount of about 1 mg/mL.

3. The immunoassay reagent of claim 1, wherein the one or more binding agent(s) is an antigen.

4. The immunoassay reagent of claim 3, wherein the antigen is a Hepatitis C virus recombinant antigen.

5. The immunoassay reagent of claim 4, wherein the Hepatitis C virus recombinant antigen is NS5, c22-3, c200, or a combination thereof.

6. The immunoassay reagent of claim 1, further including a conjugate reagent.

7. The immunoassay reagent of claim 6, wherein the conjugate reagent is HRP.

8. The immunoassay reagent of claim 1, further including an anti-microbial agent.

9. The immunoassay reagent of claim 8, wherein the anti-microbial agent is ProClin 300.

10. A sample composition comprising:
a sample to be assayed for the presence of Hepatitis C virus; and the immunoassay reagent of claim 1.

11. The sample composition of claim 10, wherein the sample is a serum sample.

12. The sample composition of claim 10, wherein the sample is a plasma sample containing ethylenediamine tetraacetic acid.

13. The sample composition of claim 10, wherein the binding agent is an antigen.

14. A method of detecting Hepatitis C virus in a sample, the method comprising:
combining a sample to be assayed for the presence of Hepatitis C virus with the immunoassay reagent of claim 1, so as to form a complex of any Hepatitis C virus present in the sample and the one or more binding agent(s), wherein the lithium heparin reduces non-specific binding in the method; and
detecting the resulting complex so as to detect Hepatitis C virus.
